SEQUENCE LISTING (4 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - DNA, 8021 bp, Homo sapiens
attctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggtaggagagcggccgcgcagacctctcgcctgctcctgcccaggggcccgccagggccatgtgagcttgaggttcccctggagtctcagccggagacaacagaagaaccgcttactgaaactccttgggggttctgatacactagggggagttttatgggaaagaggaagcagtaattgcagtgacgccccgttagaaggggctttctacctccccagcattcccccaaagcagggaccacaccattcttgacccagctccacccctgtcggtaggtgctggcttcttcccctctcctggtggtggtgggtggttcccgcggcggcctggagccggaggggcgcgcgaccctgggctgggagctccgagggcctgggaacgagacctgagaccttggcttctcgaaggtagtagggacttgggagtggtgactgaacctggtctggctcctccttacttcctcttgttgcgggtgggacgagctagcttccgcctctcccagccactttttcctgctcatttgcagctaggttggctccccttttgggaatttcctctccccttggcactcggagttggggggtgccacctagtggaagataacggagctagggtcttgaagaggctgctgtcccctctggctgttttggcggtgtagggtggcatgagagactgcgactcgcctcctcatccctgtttctgtatgcgagtgcttgtattcagtagaagcatacactatactccctcaatttagggtaaacaggaggggccacatgcacaggtaattcaccagggagccgaacactcctgtgcagacagactccccttcccagcaagccatggcagcggacagcctgctgagaacacccaggaagcaggcggtgccagctgcaggtgctttgcctgggagctgtggggctgaggagagggtccactgtccaggaccagtgaacttcatccttatctgtccaggaggtggcctcttggggatgctgagttaggggaggggcacttgaggaaagccaggtggagcagagaggatgtgagtgactgggtgggtgagatttcctgcccctccccccgcagtggtatccacacctagactcgtggggtaactgaggcacagacagagagcaacttctcaggccctcacagttggcaattctaggattaggacccaagtgcgattttcaggcagtccctgtaccctgtttctgttgtacctgttgcaccattcccaggcactgcccatcgtgccactagtgatatgaacccaggtccaatacgctctggggccatcaaagcctgacgtcaccatgacctgatgtgtgacgtgttataggtgtcccttggtatcttcacggaactggttccaggaccccaaaatctgtgggtgctcaagcccctgagataaaatggtgtaatatttgcatataacctatacatactttaaatcatttctagattacttatacctaatacaatggaaatgacatgtcggctgggcgtggtggctcatgcctgtaatcccaccactttgggaggccgtggcaggtggatcacctgaggtctggagtttgagaccagcctgaccaacatggtgaaacccccatctctactaaaaatacaaaaattagccaggtgtggtagcgcacacctataatcccacctacttgggaggctgaggcaggagaattgcttgaacctgggaggcggagttcgcagtaagctgagatcgcgccactgtactacagcctgggtgacagagcaggactccatctcaaaaaaaaaagagaaaaagaaaaagaaatgccatgtaaatagttgtgatcctgaattgtttagggaataataagaaagaactatctgtagatgttcagtatagatgcacccatcgtaagcctaactacattgtataactcagcaacgatgtaacattttcaggggtttttttgttttgttttttgagacagaatctcagtctcactctgtcacccaggctggagtatgttggcgtgatctctgctcactgcaacctccacctcctgggctcaagcgattctcctgcctcagcctcttgagtagctgggattgcaggtgtgcgctaccacgcatggctaatttttgtatttttaatagagatggggttttaccacgttggtcaggctggtcttgaactcctgaccttgggatccgcccacctgggcctcccaaagtgctgggattacaggcgttagccaccgcgcccaatatattttgatccctggttggatatggagggctgactgtacttaacatctctaagcttcagtttcctcctttaaaataaaggtgtggctgggtgtggtggttcaagcctgtaatcccagcacttagggaggctgaggtgggtggatcagctgaggtcaggagttcaagaccagcctgaccaatatggtgaaaccccctctctgctaaaaatacaaaaattagccaggcgtggtggcgagcgcctgtagtcccagctacttgcttgaacttgggaggcagaggttgcagtgagctgagatcgtgccactgaactcgagcatgggcaacagagcaagactgtctcaaaaaaaaaaaaaaaaagggggtgagcagacgtggtggcacgctcccacagtcccagctacttagtaggaggccaaggttggaggattgcttgatcccaggagtctgagtccagcctgggcaacatggcaatacctcatctctaaaaataaaataaaagtaaaggtattaattactactttggatggttgttgcaaagaaatatatataaaataatggagagtcttgtaactggctcccaagaggctcaacagacattactgtttttgcttcttcattatgagttacctctctggccaccccactgaactagctgggctagctgagcctgggagaagagttgtttaggaagtgagaggctgctctccacagagactcaaggctcagttcctcctggtgactcagatgggcagcccagtgggcacacgtggtctctctccacatgtggctgagtttcacttccagaatagatggagaggcaagggcagggtttagcatgcttgaggaatctcagagggccctggtggtgtgggggaccctcagaacacaggtgtctcaagggctgacccagcttctgtgtccttttctctgggtgaggaggggacattcatgggcagatggtgacctctggggaaggcagcccagactccactggccaccatatttcctttttcacaactttctcacccctgtggtttcccatgtcatcatgtggccgcttcccgcaaggccttagcggggtgcaggtatgaacatagtgtcaggcaaggaggcatctggaggggaaccctggcttttcctggggggactccctccctgcaccctagccctgtcctctcccatggctactgatgccttcccctcaccccagaggtggcccacatctgcacagatcagacccacaaaaatcacgtcttcctgactctcataagcctgcccagtgaggcccaggcattaggccatgtgctggggactcagacccacacatatacgcatgtcagcattcatgcttacaggtccgcacatgctggggcaagtgtcacacacggggcgctgtaggaagctgactctcagcccctgcagatttctgcctgcctggacagggaggtgttgagaaggctcaggcagtcctgggccaggaccttggcctggggctagggtactgagtgaccctagaatcaagggtggcgtgggcttaagcagttgccagacgttccttggtactttgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggtgagtgcccctcagcctaggcaagagctggcagcctgggttttcccaaagggtcatcttggattggccagaggaggacgccaggcacaagtctgtggtttatcattttccctgtctttctaggacaaatggcccacaacactgagcaggcatctgggtggcccctgccaggttgatgcccactgctctgccggccactcctgcatctttaccgtctcagggacttccagttgctgccccttcccagaggtgagcgtgccatcagcccagtggaggggcttaggtctgcatttatgcttttcctgcactctaccacctgcagataaaagggccctgccaatgcaggtttctctgtgttccacaggccgtggcatgcggggatggccatcactgctgcccacggggcttccactgcagtgcagacgggcgatcctgcttccaaagatcaggtgcagctggggtgtgggtgcagggcaggcagacgggcagcatgtggagtctggaacccaggagcccagctggcgggggcagccctgattcctgcccttgtgccctcattcatgtggcatctgtactaagcaacagccctgctgtggacagaggggcagcactggggataggagggtgcgggagaaagtgcaagactccaggtccaggcgttgtgggggtggggagaggtcgagctgggccggtctaataccaacccatggtcagtgggtgccccttccccatgccatcttgctgagggagggactggattgtgaggagggtgagttaggcctgcctaggagatcactgagccttagtgtcaccctcaaaccccagtagctgggcttgcaggccctggtgccaccagctccttgtgtgatgggggagtcaccttccctgagtgggctggtagtatcctgggtcatcttgtccacaggtaacaactccgtgggtgccatccagtgccctgatagtcagttcgaatgcccggacttctccacgtgctgtgttatggtcgatggctcctgggggtgctgccccatgccccaggtacaaatctgggggagatgggggtatgtggagggaagtgggggcagagttgggggccaggggcagggggtgaagacggagtcaggaccattttttctcaggcttcctgctgtgaagacagggtgcactgctgtccgcacggtgccttctgcgacctggttcacacccgctgcatcacacccacgggcacccaccccctggcaaagaagctccctgcccagaggactaacagggcaggtgaggaggtgggagagcatcaggccaggggctggggcggggcctcattgactccaagtgtaggaaaaagtttcctccatcctggctgcccctcacgtttgctcctcttccagtggccttgtccagctcggtcatgtgtccggacgcacggtcccggtgccctgatggttctacctgctgtgagctgcccagtgggaagtatggctgctgcccaatgcccaacgtgagtgaggggctggagccagcttggctgtgtgcccccagccacctggccctgacacgcaccttacaggggctctgtggcatggggctggctggctgcttgctgggagcctggctgatgcagggttcatgctaccccctagtgggggattggggcagtgccagccatcagcctggctgctccctgtgtgctactgagcctggaagtgacaaagacccacccctgtccccactcaggccacctgctgctccgatcacctgcactgctgcccccaagacactgtgtgtgacctgatccagagtaagtgcctctccaaggagaacgctaccacggacctcctcactaagctgcctgcgcacacaggtaccagaggcagggtgcagatacaggggtggggccccctttcctcccttttaggcctggccttaggatcactgcaaggtggtgtaagcggtaccctccatcttcaacacctggttccagctgtggagccggcaaagggttgatacccctgagggtccccagtgccacttctgacctgtcctctctgcttccctcacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggtatatgggaggggacagcatcttggcctgggcaggtgggtggccaagctcctattgctttctgccctccgcatagcccataggtgatacccagctctgacagattcgtccccagctggaggtgctgtaagcaggagaggcgggctggagtaggtaggggctcggcactgcgccccacatagtggctacctacaacgccctttcctgcccaccccccaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgtgagtgcctccctgcctgcccctggataggggagctaagcccagtgaggggacaggaacataatgccattctgtgctcccttccccgccaggctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccaggtcagtgccaacccccatcctggggctgggtatggccagggaccaggtcccacctcgtccaaccctctcgcccccctctgaccatccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaa

SEQ ID NO: 3 - DNA, 1672 bp, Artificial Sequence
  Description of Artificial Sequence Listing CAG promoter
atagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 4 - DNA, 576 bp, Cytomegalovirus
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

(19) United States
(12) Patent Application Publication
(10) Pub. No.: US 2019/0328906 A1
(43) Pub. Date: Oct. 31, 2019

(54) THERAPY FOR FRONTOTEMPORAL DEMENTIA

(71) Applicants: The Children's Hospital of Philadelphia, Philadelphia, PA (US); The Trustees of the University of Pennsylvania, Philadelphia, PA (US)

(72) Inventors: Alice CHEN PLOTKIN, Philadelphia, PA (US); Defne AMADO, Philadelphia, PA (US); Julianne REIDERS, Philadelphia, PA (US); Beverly L. DAVIDSON, Philadelphia, PA (US)

(73) Assignees: The Children's Hospital of Philadelphia, Philadelphia, PA (US); The Trustees of the University of Pennsylvania, Philadelphia, PA (US)

(21) Appl. No.: 16/081,346

(22) PCT Filed: Mar. 2, 2017
(86) PCT No.: PCT/US17/20397

§ 371 Date: Aug. 30, 2018

Related U.S. Application Data
(60) Provisional application No. 62/302,525, filed on Mar. 2, 2016.

(51) Int. Cl. A61K 48/00; A61P 25/28

(52) U.S. Cl. CPC A61K 48/005; A61P 25/28; A61K 48/0075

(57) ABSTRACT

The invention provides methods and uses for delivering progranulin to the central nervous system (CNS) of a mammal. Methods and uses include, for example, administering to a mammal a vector comprising a nucleic acid encoding progranulin, variant, derivative or functional fragment thereof to the mammal's brain ventricle to transduce CNS cells and/or cells that contact the cerebrospinal fluid (CSF) of the mammal such that the cells express the progranulin, variant, derivative or functional fragment thereof.

### RELATED APPLICATIONS

This patent application is the National Phase of International Application No. PCT/US2017/020397, filed Mar. 2, 2017, which designated the U.S. and that International Application was published under PCT Article 21(2) in English, which claims the benefit of priority to U.S. Provisional Patent Application No. 62/302,525, filed Mar. 2, 2016. The entire contents of the foregoing applications are incorporated herein by reference in their entirety, including all text, tables, sequence listing and drawings.

### SEQUENCE LISTING

The instant application contains a Sequence Listing which has been submitted electronically in ASCII format and is hereby incorporated by reference in its entirety. Said ASCII copy, created on Aug. 29, 2018, is named “CHOP0461124_ST25.txt” and is 18.8 KB in size.

### INTRODUCTION

Frontotemporal dementia (FTD) is the second most common form of early-onset dementia after Alzheimer's disease, affecting slightly more men than women in late middle age with a mean age of onset of 52-58 years. The pathology that underlies this highly heritable clinical syndrome, frontotemporal lobar degeneration (FTLD), is characterized by neuronal loss and atrophy of the frontal and temporal lobes, resulting in a spectrum of clinical manifestations ranging from apathy, to deterioration of language, to profound changes in behavior including loss of impulse control and impaired social awareness.

The socioeconomic and emotional burden of FTD on families is enormous, as patients not only deteriorate during their peak earning potential but simultaneously lose the ability to empathize with caregivers. Death ensues within 2-10 years. Behavioral symptoms such as apathy or aggression can be attenuated somewhat with medications such as antidepressants or antipsychotics, but there is no disease-modifying therapy or cure for FTD.

FTLD is subdivided pathologically by the predominant protein deposited within degenerating neurons. In about half of FTLD cases, the pathologic protein is phosphorylated tau (FTLD-tau), while the other half contain ubiquitinated inclusions (FTLD-u) that are most commonly comprised of 43kD transactive response (TAR) DNA binding protein (TDP-43; FTLD-TDP), which regulates transcription. (Of note, this is also the major pathogenic protein that accumulates in amyotrophic lateral sclerosis (ALS), and these diseases are thought to exist on a spectrum.)

A major Mendelian genetic cause of FTD-TDP is a deficiency in progranulin (GRN). GRN is a 593aa secreted precursor protein that is cleaved into granulins and is involved in multiple systemic processes including inflammation, wound repair, and development. Nearly 70 GRN mutations have been identified that cause FTLD and >90% are nonsense mutations that result in a truncated GRN product, ultimately leading to GRN haploinsufficiency. It is not known how this decrease in available GRN leads to TDP-43 accumulation and subsequent disease.

There are no disease-modifying therapies for FTD, and existing efforts to develop therapy for FTD center around targeting the various proteins (TDP-43 and tau) that accumulate in the brains of people with FTD, or around altering lysosomal function. In contrast, the invention provides a molecularly specific treatment by targeting the precise molecular defect in a readily-identified group of FTD patients.

### SUMMARY

Progranulin is a secreted growth factor known for its role in biological processes such as inflammation, wound healing, and cancer, and for its neurotrophic properties. Homozygous GRN mutations cause a rare lysosomal storage disease ceroid lipofuscinosis, and progranulin localizes to intraneuronal membrane compartments, including lysosomes. GRN heterozygotes typically develop frontotemporal dementia (FTD). The invention compositions, methods and uses are directed to treatment of both homozygous and heterozygous subjects, including mammals such as humans.

GRN variants that decrease PGRN expression increase the risk of developing Alzheimer's disease (AD) and Parkinson's disease (PD) demonstrating that insufficient PGRN predisposes neurons to degeneration. Progranulin protects against amyloid β deposition and toxicity in Alzheimer's disease. GRN polymorphism may be linked to late-onset Alzheimer's disease (AD). GRN inhibits amyloid β (Aβ) deposition reducing microglial expression of GRN in AD mouse models impaired phagocytosis, increased plaque load threefold and exacerbated cognitive deficits. GRN also protected against Aβ toxicity. GRN overexpression prevented spatial memory deficits and hippocampal neuronal loss in AD mice. The protective effects of GRN indicate that GRN can be used therapeutically for multiple neurodegenerative diseases.

In accordance with the invention, there are provided methods and uses for delivering progranulin to the central nervous system of a mammal. In one embodiment, a method or use includes administering to the mammal's brain ventricle a vector comprising a nucleic acid encoding progranulin, variant, derivative or functional fragment thereof effective to transduce cells that contact the cerebrospinal fluid (CSF) of the mammal such that the cells express the progranulin, variant, derivative or functional fragment thereof in the mammal.

In accordance with the invention, there are provided methods and uses for treating a disease in a mammal caused by a deficiency or defect in progranulin expression or function. In one embodiment, a method or use includes administering to the mammal's brain ventricle an rAAV particle comprising a vector comprising a nucleic acid encoding progranulin, variant, derivative or functional fragment thereof in a manner effective to transduce cells that contact the cerebrospinal fluid (CSF) of the mammal, wherein the cell expresses the progranulin, variant, derivative or functional fragment thereof so as to treat the disease.

In accordance with the invention, there are provided methods and uses of delivering progranulin to the central nervous system of a mammal. In one embodiment, a method or use includes administering to the mammal's brain parenchyma, subarachnoid space and/or intrathecal space a vector comprising a nucleic acid encoding a progranulin, variant, derivative or functional fragment in a manner effective to transduce brain parenchyma cells or cells that contact the cerebrospinal fluid (CSF) of the mammal such that the cells express the progranulin, variant, derivative or functional fragment in the mammal.

In accordance with the invention, there are provided methods and uses of treating a disease in a mammal caused by a deficiency or defect in progranulin expression or function. In one embodiment, a method or use includes administering to the mammal's brain parenchyma, subarachnoid space and/or intrathecal space a vector comprising a nucleic acid encoding a progranulin, variant, derivative or functional fragment inserted between a pair of AAV inverted terminal repeats in a manner effective to transduce brain parenchyma cells or cells that contact the cerebrospinal fluid (CSF) of the mammal, wherein the cell expresses the progranulin, variant, derivative or functional fragment so as to treat the disease.

In certain embodiments, a vector comprises a recombinant adeno-associated virus (rAAV) particle comprising an AAV capsid protein and the nucleic acid is inserted between a pair of AAV inverted terminal repeats.

In certain embodiments, an AAV capsid protein is selected from AAV1, AAV2, AAV3, AAV4, AAV5, AAV6, AAV7, AAV8, AAV9, AAV10, AAV11, AAV12, AAV-rh74, AAV-rh10 and AAV-2i8 VP1, VP2 and/or VP3 capsid proteins, or a capsid sequence having 60% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid sequences.

In certain embodiments, one or more of the pair of ITRs comprises or consists of an AAV1, AAV2, AAV3, AAV4, AAV5, AAV6, AAV7, AAV8, AAV9, AAV10, AAV11, AAV12, AAV-rh74, AAV-rh10 or AAV-2i8 ITR, or an ITR having 60% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

In certain embodiments, a vector (e.g., AAV vector) includes an expression control element. In certain aspects, an expression control element comprises a promoter and/or an enhancer element. In certain aspects, an expression control element comprises a CMV enhancer, chicken beta actin promoter, CAG promoter and/or a sequence having 80% or more identity to CMV enhancer set forth in SEQ ID NO:4 and/or a sequence having 80% or more identity to CAG promoter set forth in SEQ ID NO:3.

In certain embodiments, a plurality of rAAV particles are administered.

In certain embodiments, rAAV particles are administered at a dose of about 1×10⁶ to about 1×10¹⁸ vg/kg; at a dose of about 0.1-5 ml of 1×10⁷-1×10¹⁶ vg/ml; at a dose of about 0.5-5 ml of 1×10⁵-1×10¹⁶ vg/ml; at a dose of about 1-5 ml of 1×10⁵-1×10¹⁶ vg/ml; at a dose of about 1-3 ml of 1×10⁷-1×10¹⁴ vg/m; or at a dose of about 1-2 ml of 1×10⁸-1×10¹³ vg/ml.

In certain embodiments, rAAV particles are administered or delivered by intraventricular injection.

In certain embodiments, rAAV particles are administered or delivered by intraparenchymal injection.

In certain embodiments, rAAV particles are administered or delivered to brain ventricle, more particularly a lateral ventricle.

In certain embodiments, rAAV particles transduce CNS cells, more particularly, ependymal, pial, endothelial, brain ventricle, meningeal, glial cells and/or neurons.

In certain embodiments, a cell (CNS cell) expresses the progranulin, variant, derivative or functional fragment thereof.

In certain embodiments, a cell (CNS cell) secretes the progranulin, variant, derivative or functional fragment thereof into the CSF.

In certain embodiments, an ependymal, pial, endothelial, brain ventricle, meningeal cell expresses the progranulin, variant, derivative or functional fragment thereof.

In certain embodiments, an ependymal, pial, endothelial, brain ventricle, meningeal cell secretes the progranulin, variant, derivative or functional fragment thereof into the CSF.

In certain embodiments, rAAV particles are administered or delivered to the mammal's brain ventricle, subarachnoid space and/or intrathecal space.

In certain embodiments, the vector (e.g., rAAV particles) are injected at a single location in the brain.

In certain embodiments, the vector (e.g., rAAV particles) are injected at 1-5 locations in the brain.

In certain embodiments, the vector (e.g., rAAV particles) are administered or delivered as a single dose to the mammal's cisterna magna intraventricular space, brain ventricle, subarachnoid space, intrathecal space or ependyma.

In certain embodiments, the vector (e.g., rAAV particles) are administered or delivered to the rostral lateral ventricle; and/or caudal lateral ventricle; and/or right lateral ventricle; and/or left lateral ventricle; and/or right rostral lateral ventricle; and/or left rostral lateral ventricle; and/or right caudal lateral ventricle; and/or left caudal lateral ventricle.

In certain embodiments, the vector (e.g., rAAV particles) are administered or delivered in multiple doses to any of the mammal's cisterna magna intraventricular space, brain ventricle, subarachnoid space, intrathecal space and/or ependyma.

In certain embodiments, the progranulin, variant, derivative or functional fragment is mammalian (e.g., human, primate, horse, sheep, goat, pig, or dog).

In certain embodiments, the method or use provides or increases GRN expression or function, typically in CNS.

In certain embodiments, the transduced cells express the progranulin in any of the ventricle, lateral ventricle, frontal cortex, striatum, brain stem and/or spinal cord of said mammal.

In certain embodiments, the transduced cells express and secrete said progranulin into the CSF of said mammal.

In certain embodiments, the method or use increases GRN expression to between about 5-50% of normal GRN expression.

In certain embodiments, the method or use increases GRN expression to above 50% of normal GRN expression.

In certain embodiments, the method or use increases GRN expression to between about 5-50% of normal GRN expression in a human homozygous (GRN^{-/-}) with respect to lost or reduced GRN expression or function.

In certain embodiments, the method or use increases GRN expression to above 50% of normal GRN expression in a human heterozygous (GRN^{+/-}) with respect to lost or reduced GRN expression or function.

In certain embodiments, the method or use inhibits, decreases, or prevents neuron degeneration or death.

In certain embodiments, the method or use increases, preserves, restores or rescues neuron function, or viability.

In certain embodiments, the method or use increases, preserves, restores or rescues cortical neuron function, or viability.

In certain embodiments, the method or use inhibits, decreases, or prevents cortical neuron degeneration or death.

In certain embodiments, the method or use increases, preserves, restores or rescues cortical motor neuron function, or viability.

In certain embodiments, the method or use inhibits, decreases, or prevents cortical motor neuron degeneration or death.

In certain embodiments, the method or use stabilizes, prevents worsening or reverses frontotemporal lobar degeneration (FTLD).

In certain embodiments, the method or use improves, reduces or decreases a symptom or adverse effect of frontotemporal dementia (FTD) or Batten's disease.

In certain embodiments, the method or use stabilizes, prevents worsening or reverses a symptom or adverse effect of frontotemporal dementia (FTD) or Batten's disease.

In certain embodiments, a symptom or adverse effect comprises an early stage or late stage symptom; a behavior, personality or language symptom; and/or a cognitive symptom.

In certain embodiments, the mammal is a non-rodent mammal. In certain aspects, a non-rodent mammal is a primate, horse, sheep, goat, pig, or dog.

In certain embodiments, a primate is human. In certain aspects, a human is a child. In certain aspects, a child is from about 1 to about 4 years of age.

In certain embodiments, the mammal, primate or human exhibits a loss of or reduced endogenous GRN expression or function.

In certain embodiments, the mammal, primate or human is homozygous (GRN^{-/-}) or heterozygous (GRN^{+/-}) with respect to lost or reduced GRN expression or function.

In certain embodiments, the disease is caused by a deficiency or defect in progranulin expression or function.

In certain embodiments, the disease comprises frontotemporal dementia (FTD) or Batten's disease.

In certain embodiments, a method or use further includes administering or delivering one or more immunosuppressive agents. In certain aspects, an immunosuppressive agent is administered prior to or contemporaneously with administration or delivery of a vector (e.g., rAAV particles). In certain aspects, an immunosuppressive agent is an anti-inflammatory agent. In certain aspects, an immunosuppressive agent is cyclosporine, mycophenolate or a derivative thereof.

### DESCRIPTION OF DRAWINGS

FIG. 1 shows Human progranulin overexpression in progranulin null mice 1 month post injection.

FIG. 2 shows Human progranulin overexpression in progranulin null mice 3 months post injection.

### DETAILED DESCRIPTION

Although the mechanisms of GRN-deficiency-mediated neurodegeneration are unknown, restoring or increasing GRN expression or activity, optimally around physiologic levels is likely to prevent or halt the degenerative process. The invention therefore provides methods and uses of providing or restoring GRN expression, or activity via gene delivery. AAV-mediated gene delivery is in multiple clinical trials in multiple other diseases. In a particular embodiment, AAV-mediated delivery of GRN to a mammal deficient in GRN. For example, intracerebroventricular delivery of AAV-vector comprising GRN to a human in which GRN or activity expression is reduced compared to normal GRN or is absent.

Provided herein are methods and uses for administering to a mammal, in need of a method described herein, that would benefit from increased GRN activity or expression, e.g., in a subject that exhibits a loss of or reduced endogenous GRN expression or function. Thus, in one embodiment, GRN activity or expression is reduced compared to normal GRN or is absent in a subject.

In certain embodiments, a subject is homozygous (GRN^{-/-}) or heterozygous (GRN^{+/-}) with respect to lost or reduced GRN expression or function. In additional embodiments, a method or use described herein is used to treat, prevent, inhibit, reduce, decrease or delay the number, severity, frequency, progression or onset of one or more symptoms of frontotemporal dementia (FTD) or Battens disease.

In certain embodiments, provided herein are methods of treating a disease in mammal caused by a deficiency or defect in GRN activity or expression by administering, directly to a tissue or fluid of the central nervous system, a vector, such as rAAV particles that direct the expression of protein having GRN activity (referred to herein as rAAV-GRN particles). Disclosed herein are data showing rAAV-GRN delivery/administration to the brain and/or spinal cord in an animal model is effective to provide expression of GRN in various regions of the brain/CNS.

In certain embodiments, rAAV-GRN particles are administered to the brain. In certain embodiments, rAAV-GRN particles are administered to the cerebral spinal fluid (CSF) of said mammal.

In certain embodiments, rAAV-GRN particles are administered to the ventricular system. In certain embodiments, rAAV-GRN particles are administered to the brain ventricle.

In certain embodiments, rAAV-GRN particles are administered to the brain parenchyma, subarachnoid space and/or intrathecal. In certain embodiments, rAAV-GRN particles are administered to the cisternae magna, intraventricular space, subarachnoid space, intrathecal space and/or ependyma of said mammal.

In still further embodiments, rAAV-GRN particles are administered to the rostral lateral ventricle; and/or administered to the caudal lateral ventricle; and/or administered to the right lateral ventricle; and/or administered to the left lateral ventricle; and/or administered to the right rostral lateral ventricle; and/or administered to the left rostral lateral ventricle; and/or administered to the right caudal lateral ventricle; and/or administered to the left caudal lateral ventricle.

In still additional embodiments, rAAV-GRN P1 particles are administered such that the AAV particles contact and transduce CNS cells, such as ependymal cells of said mammal. Such CNS cells (e.g., ependymal cells) express the encoded GRN and optionally the GRN is secreted by the cells. In particular embodiments, the GRN is expressed and/or in CSF, brain (e.g., striatum, thalamus, medulla, cerebellum, occipital cortex, frontal cortex and/or prefrontal cortex, spinal cord), and/or CNS.

Any suitable mammal can be treated by a method or use described herein. Typically, a mammal is in need of a method described herein, that is suspected of having or that has a deficiency or defect in GRN activity or expression.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models used for the study of a deficiency or defect in progranulin expression or function, such as FTLD/FTD.

Subjects treated by a method or composition described herein include adults (18 years or older) and children (less than 18 years of age). Children range in age from 1-2 years old, or from 2-4, 4-6, 6-18, 8-10, 10-12, 12-15 and 15-18 years old. Children also include infants. Infants typically range from 1-12 months of age.

Adeno associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes been shown to stably integrate into host cellular genomes; possess a broad host range; transduce both dividing and non-dividing cells in vitro and in vivo and maintain high levels of expression of the transduced genes. AAV viral particles are heat stable, resistant to solvents, detergents, changes in pH, temperature, and can be column purified and/or concentrated on CsCl gradients or by other means. The AAV genome comprises a single-stranded deoxyribonucleic acid (ssDNA), either positive- or negative-sensed. In the absence of a helper virus, AAV may integrate in a locus specific manner, for example into the q arm of chromosome 19. The approximately 5 kb genome of AAV consists of one segment of single stranded DNA of either plus or minus polarity. The ends of the genome are short inverted terminal repeats (ITRs) which can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with capsid proteins. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into virus (e.g., AAV) particles. Thus, a vector “genome” refers to nucleic acid that is packaged or encapsulated by virus proteins and in the case of AAV, a capsid or capsid proteins.

The AAV virion (particle) is a non-enveloped, icosahedral particle approximately 25 nm in diameter. The AAV particle comprises an icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles.

An AAV particle is a viral particle comprising an AAV capsid. In certain embodiments the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments an AAV (e.g., a rAAV) comprises two ITRs. In certain embodiments an AAV (e.g., a rAAV) comprises a pair of ITRs. In certain embodiments an AAV (e.g., a rAAV) comprises a pair of ITRs that flank (i.e., are at each 5′ and 3′ end) of a polynucleotide that at least encodes a polypeptide having GRN function or activity.

The term “vector” refers to small carrier nucleic acid molecule, a plasmid, virus (e.g., AAV vector), or other vehicle that can be manipulated by insertion or incorporation of a nucleic acid. Vectors such as AAV vectors can be used to introduce/transfer polynucleotides into cells, such that the polynucleotide therein is transcribed and subsequently translated by the cells.

An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), polyadenylation signal.

A viral vector is derived from or based upon one or more nucleic acid elements that comprise a viral genome. Particular viral vectors include adeno-associated virus (AAV) vectors. As disclosed herein, provided are vectors (e.g., AAV) comprising a nucleic acid sequence encoding a GRN polypeptide, variant or subsequence (e.g., a polypeptide variant or fragment having GRN enzyme activity).

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a nucleic acid (e.g., gene) encoding a GRN polypeptide is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, “recombinant” forms including polynucleotides, nucleic acids, transgenes, etc. are expressly included in spite of any such omission.

A recombinant viral “vector” or recombinant “AAV vector” is derived from the wild type genome of a virus, such as AAV by using molecular methods to remove the wild type genome from the virus (e.g., AAV), and replacing with a non-native nucleic acid, such as a GRN encoding nucleic acid sequence. Typically, for AAV one or both inverted terminal repeat (ITR) sequences of AAV genome are retained in the rAAV vector. A “recombinant” viral vector (e.g., rAAV) is distinguished from a viral (e.g., AAV) genome, since all or a part of the viral genome has been replaced with a non-native sequence with respect to the viral (e.g., AAV) genomic nucleic acid such as GRN encoding nucleic acid sequence. Incorporation of a non-native sequence therefore defines the viral vector (e.g., AAV) as a “recombinant” vector, which in the case of AAV can be referred to as a “rAAV vector.”

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is an rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 or AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., a AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of a rAAV9 particle. In certain embodiments, a method herein comprises use, administration or delivery of a rAAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

Recombinant AAV vectors that include a polynucleotide that directs the expression of a polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, 293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

A “transgene” is used herein to conveniently refer to a nucleic acid/polynucleotide that is intended or has been introduced into a cell or organism. Transgenes include any nucleic acid, such as a gene that encodes a polypeptide or protein (e.g., GRN), and are generally heterologous with respect to naturally occurring AAV genomic sequences.

The term “transduce” refers to introduction of a nucleic acid into a cell or host organism by way of a vector (e.g., an AAV particle). Introduction of a transgene into a cell by a rAAV particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded protein expressed. For gene therapy uses and methods, a transduced cell can be in a mammal.

GRN or a polypeptide having or comprising GRN activity refers to a GRN protein of a mammal, or a portion thereof, that displays at least 50%, at least 60%, at least 70%, at least 75%, at least 80%, at least 85%, at least 90%, at least 95%, or about 100% of the peptidase activity of the human GRN of SEQ ID NO:1 using a suitable assay. In certain embodiments a polypeptide having or comprising GRN activity refers to a GRN protein of a mammal, or a subsequence or variant thereof, that displays at least at least 50%, at least 60%, at least 70%, at least 75%, at least 80%, at least 85%, at least 90%, at least 95%, or about 100% of the peptidase activity of the human GRN of SEQ ID NO:1.

A polypeptide having or comprising GRN activity may comprise a truncated, mutated, chimeric, or modified form of a GRN polypeptide that retains at least partial GRN activity. A polypeptide having or comprising GRN activity may comprise a GRN protein, or a portion thereof, obtained from any suitable organism (e.g., from a mammal, from a human, from a non-human mammal, e.g., from a dog, pig, cow, or the like). In certain embodiments a polypeptide having or comprising GRN activity has at least 60% identity, at least 70% identity, at least 75% identity, at least 80% identity, at least 85% identity, at least 90% identity, at least 95% identity, at least 98% identity, or 100% identity to the GRN protein set forth in SEQ ID NO:1.

In certain embodiments a rAAV particle comprises an AAV capsid protein and a transgene/nucleic acid encoding a polypeptide having or comprising GRN activity. In certain embodiments a rAAV particle comprises an AAV capsid protein and a nucleic acid that directs the expression and/or secretion of a polypeptide having or comprising GRN activity.

A representative human GRN amino acid sequence is depicted in SEQ ID NO:1. A representative human GRN nucleic acid sequence is depicted in SEQ ID NO:2.

In certain embodiments a rAAV particle comprises an AAV capsid protein and a transgene/nucleic acid encoding a GRN polypeptide, or enzymatically active portion thereof. In certain embodiments a rAAV particle comprises an AAV capsid protein and a transgene/nucleic acid that directs the expression and/or secretion of a GRN polypeptide, or enzymatically active portion thereof. In certain embodiments, a nucleic acid being administered encodes GRN, a GRN that has substantial identity to wild type GRN, and/or a variant, mutant or fragment of a GRN. In certain embodiments a GRN polypeptide has at least 60% identity, at least 70% identity, at least 75% identity, at least 80% identity, at least 85% identity, at least 90% identity, at least 95% identity, at least 98% identity, or 100% identity to the protein set forth in SEQ ID NO:1.

In certain embodiments a rAAV particle comprises a transgene/nucleic acid having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO:2. In certain embodiments a transgene/nucleic acid encoding a protein having GRN function or activity or encoding or directing the expression of a GRN polypeptide is a nucleic acid having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO:2.

In certain embodiments a method or use includes administering or delivering rAAV-GRN particles to a mammal and optionally administering one or more immunosuppressive agents to the mammal. In certain embodiments a method or use includes administering or delivering rAAV-GRN particles to a mammal and optionally administering 2, 3, 4 or more immunosuppressive agents to the mammal.

In certain embodiments, an immunosuppressive agent is an anti-inflammatory agent. In certain embodiments, an immunosuppressive agent is mycophenolate, or a derivative thereof. An example of such a mycophenolate derivative is mycophenolate mofetil (MMF). In certain embodiments, an immunosuppressive agent is cyclosporine or a derivative thereof. Where two or more immunosuppressive agents are administered, each immunosuppressive agent is distinct and/or different (e.g., each agent differs in structure and/or mechanism of action).

In certain embodiments, an immunosuppressive agent is administered before, during and/or after administration of rAAV-GRN particles to a mammal. In certain embodiments, an immunosuppressive agent is administered concurrently with administration of rAAV-GRN particles to a mammal. In certain embodiments, an immunosuppressive agent is administered after administration of rAAV-GRN particles to a mammal.

An immunosuppressive agent can be administered at any suitable dose. In certain embodiments, cyclosporine is administered at a dosage of about 1 to about 50 mg/kg, about 1 to about 20 mg/kg, or about 5 to about 10 mg/kg at a frequency of once, twice or three times a day, to once every other day. In certain embodiments cyclosporine is administered at about 10 mg/kg twice a day. In certain embodiments, cyclosporine is administered at about 10 mg/kg twice a day for a period of at least about 1, about 2, about 3, about 4 or about 5 months.

In certain embodiments, mycophenolate or a derivative thereof (e.g., MMF), is administered at a dosage of about 1 to about 100 mg/kg, about 1 to about 50 mg/kg, about 1 to about 25 mg/kg, or about 5 to about 20 mg/kg at a frequency of once, twice or three times a day, to once every other day. In certain embodiments, mycophenolate or a derivative thereof (e.g., MMF) is administered at about 10 to about 20 mg/kg once a day.

A rAAV particle and/or immunosuppressive agent can be formulated in any suitable formulation suitable for a particular route of administration. Various pharmaceutically acceptable formulations are commercially available and obtainable by a medical practitioner.

A rAAV particle can be administered by any suitable route. In certain embodiments a method or use includes administering rAAV-GRN particles to the central nervous system (CNS) of a mammal. In certain embodiments, the central nervous system includes brain, spinal cord and cerebral spinal fluid (CSF). In certain embodiments, a method or use includes administering rAAV-GRN particles to the brain or spinal cord or CSF of a mammal. In certain embodiments, rAAV-GRN particles are administered to a portion of brain or spinal cord.

certain embodiments, rAAV-GRN particles are administered to brain parenchyma, subarachnoid space and/or intrathecal space. In certain embodiments, rAAV-GRN particles are administered to one or more of cisterna magna, intraventricular space, brain ventricle, subarachnoid space, and/or ependyma of said mammal.

In further embodiments, rAAV-GRN particles are administered to the ventricular system. In still further embodiments, rAAV-GRN particles are administered to one or more of the rostral lateral ventricle; and/or caudal lateral ventricle; and/or right lateral ventricle; and/or left lateral ventricle; and/or right rostral lateral ventricle; and/or left rostral lateral ventricle; and/or right caudal lateral ventricle; and/or left caudal lateral ventricle.

In certain embodiments rAAV-GRN particles are administered to one or more cells that contact the CSF in a mammal, for example by contacting cells with rAAV-GRN particles. Non-limiting examples of cells that contact the CSF include ependymal cells, pial cells, endothelial cells and/or meningeal cells. In certain embodiments rAAV-GRN particles are administered to ependymal cells. In certain embodiments rAAV-GRN particles are delivered to ependymal cells, for example by contacting ependymal cells with rAAV-GRN particles.

In certain embodiments, rAAV-GRN particles are administered/delivered locally. “Local delivery” refers to delivery directly to a target site within a mammal (e.g., directly to a tissue or fluid). For example, rAAV-GRN particles can be locally delivered by direct injection into an organ, tissue or specified anatomical location. In certain embodiments, rAAV-GRN particles are delivered or administered by direct injection to the brain, spinal cord, or a tissue or fluid thereof (e.g., CSF, such as ependymal cells, pial cells, endothelial cells and/or meningeal cells). For example rAAV-GRN particles can be directly delivered, by way of direct injection, to the CSF, cisterna magna, intraventricular space, a brain ventricle, subarachnoid space and/or intrathecal space; and/or ependymal; and/or rostral lateral ventricle; and/or caudal lateral ventricle; and/or right lateral ventricle; and/or left lateral ventricle; and/or right rostral lateral ventricle; and/or left rostral lateral ventricle; and/or right caudal lateral ventricle; and/or left caudal lateral ventricle.

In certain embodiments, rAAV-GRN particles are delivered to a tissue, fluid or cell of the brain or spinal cord by direct injection into a tissue or fluid of the brain or spinal cord. In certain embodiments, rAAV-GRN particles are not delivered systemically by, for example, intravenous, subcutaneous, or intramuscular injection, or by intravenous infusion. In certain embodiments, rAAV-GRN particles are delivered to a tissue or fluid of the brain or spinal cord by stereotactic injection.

In certain embodiments one or more rAAV-GRN particles are delivered or administered by direct injection to the brain, spinal cord, or portion thereof, or a tissue or fluid thereof (e.g., CSF such as ependyma). In a particular aspect, rAAV-GRN particles transduce ependymal cells, pial cells, endothelial cells and/or meningeal cells.

In certain embodiments, a rAAV particles are configured to transduce cells of the mammal and direct expression of a polypeptide having GRN activity in the mammal. In certain embodiments, the polypeptide is expressed and/or detected in one or more peripheral organs (e.g., in liver).

In certain embodiments, a method or use includes administering rAAV particles to the brain or spinal cord, or portion thereof, of a mammal where the rAAV particles are configured to transduce brain or spinal cord cells of the mammal and direct expression of the polypeptide having GRN activity in the brain or spinal cord of the mammal. In certain embodiments, the polypeptide is expressed and/or detected in a central nervous tissue (e.g., brain, e.g., striatum, thalamus, medulla, cerebellum, occipital cortex, prefrontal cortex) distal to the administration site. In certain embodiments, the polypeptide is present or detected broadly in a central nervous tissue (e.g., brain, e.g., striatum, thalamus, medulla, cerebellum, occipital cortex, and/or prefrontal cortex) that reflects distribution away from the administration site and optionally throughout a central nervous tissue (e.g., brain, e.g., striatum, thalamus, medulla, cerebellum, occipital cortex, and/or prefrontal cortex).

An effective amount of rAAV particles, such as rAAV-GRN particles, can be empirically determined. Administration can be effected in one or more doses, continuously or intermittently throughout the course of treatment. Effective doses of administration can be determined by those of skill in the art and may vary according to the AAV serotype, viral titer and the weight, condition and species of mammal being treated. Single and multiple administrations (e.g., 1-5 or more) can be carried out with the dose level, target and timing being selected by the treating physician. Multiple doses may be administered as is required to maintain adequate enzyme activity, for example.

In certain embodiments, a plurality of rAAV-GRN particles are administered. In certain embodiments, rAAV-GRN particles are administered at a dose of about 1×10⁵ to about 1×10¹⁸ vg/ml in about 1 to about 5 ml; at a dose of about 1 to about 3 ml of 1×10⁷ to about 1×10¹⁶ vg/ml; or at a dose of about 1 to about 2 ml of 1×10⁸ to about 1×10¹⁵ vg/ml. In certain embodiments, rAAV-GRN particles are administered at a dose of about 1×10⁸ to about 1×10¹⁵ vg/kg body weight of the mammal being treated.

In certain embodiments, rAAV-GRN particles are administered at a dose of about 1×10⁶ to about 1×10¹⁸ vg/kg. For example, rAAV-GRN particles can be administered at a dose of about 0.1-5 ml of 1×10⁷-1×10¹⁶ vg/ml, about 0.5-5 ml of 1×10⁵-1×10¹⁶ vg/ml, about 1-5 ml of 1×10⁵-1×10¹⁶ vg/ml, about 1-3 ml of 1×10⁷-1×10¹⁴ vg/ml or a dose of about 1-2 ml of 1×10⁸-1×10¹³ vg/ml.

In certain embodiments, rAAV-GRN particles are administered at a dose of about 1×10⁸ vg/kg, about 5×10⁸ vg/kg, about 1×10⁹ vg/kg, about 5×10⁹ vg/kg, about 1×10¹⁰ vg/kg, about 5×10¹⁰ vg/kg, about 1×10¹¹ vg/kg, about 5×10¹¹ vg/kg, about 1×10¹² vg/kg, about 5×10¹² vg/kg, about 1×10¹³ vg/kg, about 5×10¹³ vg/kg, about 1×10¹⁴ vg/kg, about 5×10¹⁴ vg/kg, or about 1×10¹⁵ vg/kg body weight of the mammal being treated.

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a rAAV-GRN particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of rAAV particles, such as rAAV-GRN particles particles, can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of rAAV-GRN particles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

rAAV particles, such as rAAV-GRN particles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of rAAV particles (e.g., rAAV-GRN particles) believed necessary to produce the desired effect(s). The amount necessary can be formulated in a single dose, or can be formulated in multiple dosage units. The dose may be adjusted to a suitable rAAV particles concentration, optionally combined with an anti-inflammatory agent, and packaged for use.

In one embodiment, pharmaceutical compositions will include sufficient genetic material (rAAV particles) to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, rAAV-GRN particles, and pharmaceutical compositions thereof can be packaged in single or multiple unit dosage form for ease of administration and uniformity of dosage.

Formulations containing rAAV-GRN particles typically contain an effective amount, the effective amount being readily determined by one skilled in the art. The rAAV-GRN particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

In certain embodiments a method includes administering a plurality of rAAV-GRN particles to a mammal as set forth herein, where severity, frequency, progression or time of onset of one or more symptoms of a deficiency or defect in progranulin expression or function (e.g., FTD/FTLD) are decreased, reduced, prevented, inhibited or delayed. In certain embodiments a method includes administering a plurality of rAAV-GRN particles to a mammal to treat a symptom or adverse effect of frontotemporal dementia (FTD) or Batten's disease. In certain embodiments a method includes administering a plurality of rAAV-GRN particles to a mammal to stabilize, delay or prevent worsening, or reverse a symptom or adverse effect of frontotemporal dementia (FTD) or Batten's disease.

In certain embodiments a method includes administering a plurality of AAV-GRN particles to the central nervous system, or portion thereof as set forth herein, of a mammal and severity, frequency, progression or time of onset of one or more symptoms of a deficiency or defect in progranulin expression or function (e.g., FTD/FTLD) are decreased, reduced, prevented, inhibited or delayed by at least about 5 to about 10, about 10 to about 25, about 25 to about 50, or about 50 to about 100 days.

In certain embodiments, a symptom or adverse effect comprises an early stage or late stage symptom; a behavior, personality or language symptom; and/or a cognitive symptom.

Examples of early symptoms/adverse effects of FTD treatable according to the methods and uses herein include improvements or slowing or preventing progression or worsening of personality or mood changes such as depression and withdrawal, sometimes obsessive behavior and language difficulties. Many FTD subjects lose their inhibitions and exhibit antisocial and/or aggressive behavior. Such symptoms include apathy or an unwillingness to talk; change in personality and mood, such as depression; lack of inhibition or lack of social tact; obsessive or repetitive behavior, such as compulsively shaving or collecting items; unusual verbal, physical or sexual behavior; and weight gain due to dramatic overeating. FTD subjects may neglect hygiene and resist encouragement to attend to themselves. They also may lack awareness or concern that their behavior has changed.

Some FTD subjects develop extraordinary visual or musical creativity, while experiencing language and social impairment. Artistic talents developed when brain cell loss occurred predominantly in the left frontal lobe, which controls functions such as language. It is believed that the right side of the brain regulates more abstract reasoning.

Examples of other symptoms/adverse effects of FTD treatable according to the methods and uses herein include improvements or slowing or preventing progression or worsening of language problems, which are less common but do occur in early stages of FTD before other thought processes, such as memory, are affected. FTD subjects may experience difficulty speaking or finding the correct word when naming objects. Difficulties reading and writing can then develop. As the disease progresses, less and less language is used, until they become virtually mute. Other FTD subjects may have severe problems recalling words and understanding word meaning, but continue to have otherwise normal speech.

Examples of other symptoms/adverse effects of FTD treatable according to the methods and uses herein include improvements or slowing or preventing progression or worsening of FTD progression, which affects cognitive/mental abilities, such as memory and other functions that are more common in Alzheimer's disease and other dementias. In Alzheimer's, one of the first symptoms is memory loss. With FTD, unusual or antisocial behavior as well as loss of speech or language are usually the first symptoms.

Examples of later stage symptoms/adverse effects of FTD treatable according to the methods and uses herein include improvements or slowing or preventing progression or worsening of movement disorders such as unsteadiness, rigidity, slowness, twitches, muscle weakness or difficulty swallowing. Some patients develop Lou Gherig's disease or amyotrophic lateral sclerosis (ALS). People in the final stages of FTD cannot care for themselves.

FTD diagnosis requires a physical examination. In particular, a method in which brain tissue loss can be detected, such as by imaging tests. Exemplary non-limiting tests include magnetic resonance images (MRI), which can identify the characteristic shrinking of the frontal and temporal lobes, located in the front of the brain. Other tests include, but are not limited to, positron emission tomography (PET), computed tomography (CT) and single photon emission computed tomography (SPECT). Accordingly, the foregoing can be used to diagnose as well as determine treatment efficacy, such as an improvement, or slowing or preventing progression or worsening of one or more symptoms of a deficiency or defect in progranulin expression or function (e.g., FTD/FTLD).

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid/transgene is “operably linked” when it is placed into a functional relationship with another nucleic acid sequence. A nucleic acid/transgene encoding a polypeptide, or a nucleic acid directing expression of a GRN polypeptide (e.g., a polypeptide having GRN activity) may include an inducible promoter, or a tissue-specific promoter for controlling transcription of the encoded polypeptide.

In certain embodiments, CNS-specific or inducible promoters, enhancers and the like, are employed in the methods and uses described herein. Non-limiting examples of CNS-specific promoters include those isolated from the genes from myelin basic protein (MBP), glial fibrillary acid protein (GFAP), and neuron specific enolase (NSE). Non-limiting examples of inducible promoters include DNA responsive elements for ecdysone, tetracycline, hypoxia and IFN.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

In certain embodiments, an expression control element comprises a sequence having 80% or more identity to CMV enhancer set forth in SEQ ID NO:4 and/or a sequence having 80% or more identity to CAG promoter set forth in SEQ ID NO:3. In certain embodiments, an expression control element comprises SEQ ID NO:4. In certain embodiments, an expression control element comprises SEQ ID NO:3.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation in GRN.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein, e.g., a variant GRN protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a GRN protein encoded thereby, and some codons of the nucleic acid variant will be changed which in turn changes the amino acids of a GRN protein encoded thereby.

The terms “protein” and “polypeptide” are used interchangeably herein. The “polypeptides” encoded by a “nucleic acid” or “polynucleotide” or “transgene” disclosed herein include partial or full-length native GRN sequences, as with naturally occurring wild-type and functional polymorphic proteins, functional subsequences (fragments) thereof, and modified forms or sequence variants thereof, so long as the polypeptide retains some degree of GRN activity. Accordingly, in methods and uses of the invention, such polypeptides encoded by nucleic acid sequences can be, but are not required to be, identical to the endogenous GRN protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues). One non-limiting example of a nucleic acid modification is codon optimization.

An example of an amino acid modification is a conservative amino acid substitution or a deletion. In particular embodiments, a modified or variant sequence (e.g., GRN) retains at least part of a function or activity of the unmodified sequence (e.g., wild-type GRN).

Another example of an amino acid modification is a targeting peptide introduced into a capsid protein of an AAV particle. Peptides have been identified that target rAAV vectors, to the central nervous system, such as vascular endothelial cells. Thus, for example, endothelial cells lining brain blood vessels can be targeted by the modified rAAV particles. rAAV-GRN particle bearing capsid proteins modified to include such peptides can be used to introduce GRN into the central nervous system (e.g., the brain, spinal cord, etc.) as set forth herein.

A rAAV so modified may preferentially bind to one type of tissue (e.g., CNS tissue) over another type of tissue (e.g., liver tissue). In certain embodiments, a rAAV bearing a modified capsid protein may “target” brain vascular epithelia tissue by binding at level higher than a comparable, unmodified capsid protein. For example, a rAAV having a modified capsid protein may bind to brain vascular epithelia tissue at a level 50% to 100% greater than an unmodified rAAV.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type GRN).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type GRN).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are substantially identical is that one polypeptide is immunologically reactive with antibodies raised against the second polypeptide. Thus, a polypeptide is substantially identical to a second polypeptide, for example, where the two peptides differ only by a conservative substitution.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, rAAV-GRN particles and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

The term “about” at used herein refers to a values that is within 10% (plus or minus) of a reference value.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay), such as those identified to be homozygous (GRN^{-/-}) with respect to lost or reduced GRN expression or function or heterozygous (GRN^{+/-}) with respect to lost or reduced GRN expression or function.

The terms “comprising,” “having,” “including,” and “containing” are to be construed as open-ended terms (i.e., meaning “including, but not limited to”) unless otherwise noted.

All methods and uses described herein can be performed in any suitable order unless otherwise indicated herein or otherwise clearly contradicted by context. The use of any and all examples, or exemplary language (e.g., “such as” or “for example”) provided herein, is intended merely to better illuminate the invention and does not pose a limitation on the scope of the invention unless otherwise claimed. No language in the specification should be construed as indicating any non-claimed element as essential to the practice of the invention.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified nucleic acid, vector, plasmid, a recombinant vector (e.g., rAAV) sequence, vector genome, or rAAV particle) are an example of a genus of equivalent or similar features.

As used herein, the forms “a”, “and,” and “the” include singular and plural referents unless the context clearly indicates otherwise. Thus, for example, reference to “a nucleic acid” includes a plurality of such nucleic acids, reference to “a vector” includes a plurality of such vectors, and reference to “a virus” or “AAV or rAAV particle” includes a plurality of such virions/AAV or rAAV particles.

Recitation of ranges of values herein are merely intended to serve as a shorthand method of referring individually to each separate value falling within the range, unless otherwise indicated herein, and each separate value is incorporated into the specification as if it were individually recited herein.

Accordingly, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-20, 10-50, 30-50, 50-100, 100-300, 100-1,000, 1,000-3,000, 2,000-4,000, 4,000-6,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

FTD is the second-most common cause of dementia in individuals younger than 65 years of age, and mutations in the gene encoding progranulin (GRN) are a common Mendelian cause of FTD. To date, all mutations (>50 different mutations) in GRN that cause FTD have been shown to do so by haploinsufficiency—the vast majority are nonsense mutations causing the affected individual to express only 50% of normal transcript levels. As a consequence, replacement of progranulin should provide a therapy to ameliorate, reverse, or even prevent FTD due to GRN mutations.

AAV viral vectors will be used to express the progranulin gene (GRN) in the central nervous system (CNS) as a therapy for frontotemporal dementia (FTD). Multiple AAV vectors are developed that can deliver human progranulin. As disclosed herein, AAV vectors can deliver measurable amounts of progranulin to the CNS by administration routes such as intraparenchymal or intraventricular injection in studies involving mice. A series of studies in mice lacking the GRN gene to verify that AAV-GRN introduction into the ventricles can rescue phenotypes associated with GRN deficiency in these animals.

Progranulin is also called acrogranin, CLN11, GEP, GP88, Granulin, granulin-epithelin, granulins, granulins precursor, GRN_HUMAN, PC cell-derived growth factor, PCDGF, PEPI, PGRN, and Proepithelin.

Exemplary human progranulin (GRN) protein (SEQ ID NO: 1):
MWTLVSWVALTAGLVAGTRCPDGQFCPVACCLDPGGASYSCCRPLLDKWPTTLSRHLGGPCQVDAHCSAGHSCIFTV
SGTSSCCPFPEAVACGDGHHCCPRGFHCSADGRSCFQRSGNNSVGAIQCPDSQFECPDFSTCCVMVDGSWGCCPMPQ
ASCCEDRVHCCPHGAFCDLVHTRCITPTGTHPLAKKLPAQRTNRAVALSSSVMCPDARSRCPDGSTCCELPSGKYGC
CPMPNATCCSDHLHCCPQDTVCDLIQSKCLSKENATTDLLTKLPAHTVGDVKCDMEVSCPDGYTCCRLQSGAWGCCP
FTQAVCCEDHIHCCPAGFTCDTQKGTCEQGPHQVPWMEKAPAHLSLPDPQALKRDVPCDNVSSCPSSDTCCQLTSGE
WGCCPIPEAVCCSDHQHCCPQGYTCVAEGQCQRGSEIVAGLEKMPARRASLSHPRDIGCDQHTSCPVGQTCCPSLGG
SWACCQLPHAVCCEDRQHCCPAGYTCNVKARSCEKEVVSAQPATFLARSPHVGVKDVECGEGHFCHDNQTCCRDNRQ
GWACCPYRQGVCCADRRHCCPAGFRCAARGTKCLRREAPRWDAPLRDPALRQLL
Exemplary nucleic acid encoding human GRN protein (SEQ ID NO: 2):

| | |
|---|---|
| 1 | attctccaat cacatgatcc ctagaaatgg ggtgtggggc gagaggaagc agggaggaga |
| 61 | gtgatttgag tagaaaagaa acacagcatt ccaggctggc cccacctcta tattgataag |
| 121 | tagccaatgg gagcgggtag ccctgatccc tggccaatgg aaactgaggt aggcgggtca |
| 181 | tcgcgctggg gtctgtagtc tgagcgctac ccggttgctg ctgcccaagg accgcggagt |
| 241 | cggacgcagg taggagagcg gccgcgcaga cctctcgcct gctcctgccc aggggcccgc |
| 301 | cagggccatg tgagcttgag gttcccctgg agtctcagcc ggagacaaca gaagaaccgc |
| 361 | ttactgaaac tccttggggg ttctgataca ctagggggag ttttatggga aagaggaagc |
| 421 | agtaattgca gtgacgcccc gttagaaggg gctttctacc tccccagcat tcccccaaag |
| 481 | cagggaccac accattcttg acccagctcc acccctgtcg gtaggtgctg gcttcttccc |
| 541 | ctctcctggt ggtggtgggt ggttcccgcg gcggcctgga gccggagggg cgcgcgaccc |
| 601 | tgggctggga gctccgaggg cctgggaacg agacctgaga ccttggcttc tcgaaggtag |
| 661 | tagggacttg ggagtggtga ctgaacctgg tctggctcct ccttacttcc tcttgttgcg |
| 721 | ggtgggacga gctagcttcc gcctctccca gccacttttt cctgctcatt tgcagctagg |
| 781 | ttggctcccc ttttgggaat ttcctctccc cttggcactc ggagttgggg ggtgccacct |
| 841 | agtggaagat aacggagcta gggtcttgaa gaggctgctg tcccctctgg ctgttttggc |
| 901 | ggtgtagggt ggcatgagag actgcgactc gcctcctcat ccctgtttct gtatgcgagt |
| 961 | gcttgtattc agtagaagca tacactatac tccctcaatt tagggtaaac aggaggggcc |
| 1021 | acatgcacag gtaattcacc agggagccga acactcctgt gcagacagac tccccttccc |
| 1081 | agcaagccat ggcagcggac agcctgctga gaacacccag gaagcaggcg gtgccagctg |
| 1141 | caggtgcttt gcctgggagc tgtggggctg aggagagggt ccactgtcca ggaccagtga |
| 1201 | acttcatcct tatctgtcca ggaggtggcc tcttggggat gctgagttag gggaggggca |
| 1261 | cttgaggaaa gccaggtgga gcagagagga tgtgagtgac tgggtgggtg agatttcctg |
| 1321 | cccctccccc cgcagtggta tccacaccta gactcgtggg gtaactgagg cacagacaga |
| 1381 | gagcaacttc tcaggccctc acagttggca attctaggat taggacccaa gtgcgatttt |
| 1441 | caggcagtcc ctgtaccctg tttctgttgt acctgttgca ccattcccag gcactgccca |
| 1501 | tcgtgccact agtgatatga acccaggtcc aatacgctct ggggccatca aagcctgacg |
| 1561 | tcaccatgac ctgatgtgtg acgtgttata ggtgtccctt ggtatcttca cggaactggt |
| 1621 | tccaggaccc caaaatctgt gggtgctcaa gcccctgaga taaaatggtg taatatttgc |
| 1681 | atataaccta tacatacttt aaatcatttc tagattactt atacctaata caatggaaat |
| 1741 | gacatgtcgg ctgggcgtgg tggctcatgc ctgtaatccc accactttgg gaggccgtgg |
| 1801 | caggtggatc acctgaggtc tggagtttga gaccagcctg accaacatgg tgaaaccccc |
| 1861 | atctctacta aaaatacaaa aattagccag gtgtggtagc gcacacctat aatcccacct |
| 1921 | acttgggagg ctgaggcagg agaattgctt gaacctggga ggcggagttc gcagtaagct |
| 1981 | gagatcgcgc cactgtacta cagcctgggt gacagagcag gactccatct caaaaaaaaa |
| 2041 | agagaaaaag aaaaagaaat gccatgtaaa tagttgtgat cctgaattgt ttagggaata |
| 2101 | ataagaaaga actatctgta gatgttcagt atagatgcac ccatcgtaag cctaactaca |
| 2161 | ttgtataact cagcaacgat gtaacatttt caggggtttt tttgttttgt tttttgagac |
| 2221 | agaatctcag tctcactctg tcacccaggc tggagtatgt tggcgtgatc tctgctcact |
| 2281 | gcaacctcca cctcctgggc tcaagcgatt ctcctgcctc agcctcttga gtagctggga |
| 2341 | ttgcaggtgt gcgctaccac gcatggctaa tttttgtatt tttaatagag atggggtttt |
| 2401 | accacgttgg tcaggctggt cttgaactcc tgaccttggg atccgcccac ctgggcctcc |
| 2461 | caaagtgctg ggattacagg cgttagccac cgcgcccaat atattttgat ccctggttgg |
| 2521 | atatggaggg ctgactgtac ttaacatctc taagcttcag tttcctcctt taaaataaag |
| 2581 | gtgtggctgg gtgtggtggt tcaagcctgt aatcccagca cttagggagg ctgaggtggg |
| 2641 | tggatcagct gaggtcagga gttcaagacc agcctgacca atatggtgaa accccctctc |
| 2701 | tgctaaaaat acaaaaatta gccaggcgtg gtggcgagcg cctgtagtcc cagctacttg |
| 2761 | cttgaacttg ggaggcagag gttgcagtga gctgagatcg tgccactgaa ctcgagcatg |
| 2821 | ggcaacagag caagactgtc tcaaaaaaaa aaaaaaaaag ggggtgagca gacgtggtgg |
| 2881 | cacgctccca cagtcccagc tacttagtag gaggccaagg ttggaggatt gcttgatccc |
| 2941 | aggagtctga gtccagcctg ggcaacatgg caatacctca tctctaaaaa taaaataaaa |
| 3001 | gtaaaggtat taattactac tttggatggt tgttgcaaag aaatatatat aaaataatgg |
| 3061 | agagtcttgt aactggctcc caagaggctc aacagacatt actgtttttg cttcttcatt |
| 3121 | atgagttacc tctctggcca ccccactgaa ctagctgggc tagctgagcc tgggagaaga |
| 3181 | gttgtttagg aagtgagagg ctgctctcca cagagactca aggctcagtt cctcctggtg |
| 3241 | actcagatgg gcagcccagt gggcacacgt ggtctctctc cacatgtggc tgagtttcac |
| 3301 | ttccagaata gatggagagg caagggcagg gtttagcatg cttgaggaat ctcagagggc |
| 3361 | cctggtggtg tgggggaccc tcagaacaca ggtgtctcaa gggctgaccc agcttctgtg |
| 3421 | tccttttctc tgggtgagga ggggacattc atgggcagat ggtgacctct ggggaaggca |
| 3481 | gcccagactc cactggccac catatttcct ttttcacaac tttctcaccc ctgtggtttc |
| 3541 | ccatgtcatc atgtggccgc ttcccgcaag gccttagcgg ggtgcaggta tgaacatagt |
| 3601 | gtcaggcaag gaggcatctg gaggggaacc ctggcttttc ctggggggac tccctccctg |
| 3661 | caccctagcc ctgtcctctc ccatggctac tgatgccttc ccctcacccc agaggtggcc |
| 3721 | cacatctgca cagatcagac ccacaaaaat cacgtcttcc tgactctcat aagcctgccc |
| 3781 | agtgaggccc aggcattagg ccatgtgctg gggactcaga cccacacata tacgcatgtc |
| 3841 | agcattcatg cttacaggtc cgcacatgct ggggcaagtg tcacacacgg ggcgctgtag |
| 3901 | gaagctgact ctcagcccct gcagatttct gcctgcctgg acagggaggt gttgagaagg |
| 3961 | ctcaggcagt cctgggccag gaccttggcc tggggctagg gtactgagtg accctagaat |
| 4021 | caagggtggc gtgggcttaa gcagttgcca gacgttcctt ggtactttgc aggcagacca |
| 4081 | tgtggaccct ggtgagctgg gtggccttaa cagcagggct ggtggctgga acgcggtgcc |
| 4141 | cagatggtca gttctgccct gtggcctgct gcctggaccc cggaggagcc agctacagct |
| 4201 | gctgccgtcc ccttctggtg agtgcccctc agcctaggca agagctggca gcctgggttt |
| 4261 | tcccaaaggg tcatcttgga ttggccagag gaggacgcca ggcacaagtc tgtggtttat |
| 4321 | cattttccct gtctttctag gacaaatggc ccacaacact gagcaggcat ctgggtggcc |
| 4381 | cctgccaggt tgatgcccac tgctctgccg gccactcctg catctttacc gtctcaggga |
| 4441 | cttccagttg ctgccccttc ccagaggtga gcgtgccatc agcccagtgg aggggcttag |
| 4501 | gtctgcattt atgcttttcc tgcactctac cacctgcaga taaaagggcc ctgccaatgc |
| 4561 | aggtttctct gtgttccaca ggccgtggca tgcggggatg gccatcactg ctgcccacgg |
| 4621 | ggcttccact gcagtgcaga cgggcgatcc tgcttccaaa gatcaggtgc agctggggtg |
| 4681 | tgggtgcagg gcaggcagac gggcagcatg tggagtctgg aacccaggag cccagctggc |
| 4741 | gggggcagcc ctgattcctg cccttgtgcc ctcattcatg tggcatctgt actaagcaac |
| 4801 | agccctgctg tggacagagg ggcagcactg gggataggag ggtgcgggag aaagtgcaag |
| 4861 | actccaggtc caggcgttgt gggggtgggg agaggtcgag ctgggccggt ctaataccaa |
| 4921 | cccatggtca gtgggtgccc cttccccatg ccatcttgct gagggaggga ctggattgtg |
| 4981 | aggagggtga gttaggcctg cctaggagat cactgagcct tagtgtcacc ctcaaacccc |
| 5041 | agtagctggg cttgcaggcc ctggtgccac cagctccttg tgtgatgggg gagtcacctt |
| 5101 | ccctgagtgg gctggtagta tcctgggtca tcttgtccac aggtaacaac tccgtgggtg |
| 5161 | ccatccagtg ccctgatagt cagttcgaat gcccggactt ctccacgtgc tgtgttatgg |
| 5221 | tcgatggctc ctgggggtgc tgccccatgc cccaggtaca aatctggggg agatgggggt |
| 5281 | atgtggaggg aagtgggggc agagttgggg gccaggggca gggggtgaag acggagtcag |
| 5341 | gaccattttt tctcaggctt cctgctgtga agacagggtg cactgctgtc cgcacggtgc |
| 5401 | cttctgcgac ctggttcaca cccgctgcat cacacccacg ggcacccacc ccctggcaaa |
| 5461 | gaagctccct gcccagagga ctaacagggc aggtgaggag gtgggagagc atcaggccag |
| 5521 | gggctggggc ggggcctcat tgactccaag tgtaggaaaa agtttcctcc atcctggctg |
| 5581 | cccctcacgt ttgctcctct tccagtggcc ttgtccagct cggtcatgtg tccggacgca |
| 5641 | cggtcccggt gccctgatgg ttctacctgc tgtgagctgc ccagtgggaa gtatggctgc |
| 5701 | tgcccaatgc ccaacgtgag tgaggggctg gagccagctt ggctgtgtgc ccccagccac |
| 5761 | ctggccctga cacgcacctt acaggggctc tgtggcatgg ggctggctgg ctgcttgctg |
| 5821 | ggagcctggc tgatgcaggg ttcatgctac cccctagtgg gggattgggg cagtgccagc |
| 5881 | catcagcctg gctgctccct gtgtgctact gagcctggaa gtgacaaaga cccacccctg |
| 5941 | tccccactca ggccacctgc tgctccgatc acctgcactg ctgcccccaa gacactgtgt |
| 6001 | gtgacctgat ccagagtaag tgcctctcca aggagaacgc taccacggac ctcctcacta |
| 6061 | agctgcctgc gcacacaggt accagaggca gggtgcagat acaggggtgg ggcccccttt |
| 6121 | cctccctttt aggcctggcc ttaggatcac tgcaaggtgg tgtaagcggt accctccatc |
| 6181 | ttcaacacct ggttccagct gtggagccgg caaagggttg atacccctga gggtccccag |
| 6241 | tgccacttct gacctgtcct ctctgcttcc ctcacagtgg gggatgtgaa atgtgacatg |
| 6301 | gaggtgagct gcccagatgg ctatacctgc tgccgtctac agtcgggggc ctggggctgc |
| 6361 | tgccctttta cccaggtacc caggggtggc gggtgggtgg gctgagcaca gtgtggcagg |
| 6421 | cagccgggcc ccagtgccca cctgcccttc ttcatctgcc ctaggctgtg tgctgtgagg |
| 6481 | accacataca ctgctgtccc gcggggttta cgtgtgacac gcagaagggt acctgtgaac |
| 6541 | aggggcccca ccaggtgccc tggatggaga aggccccagc tcacctcagc ctgccagacc |
| 6601 | cacaagcctt gaagagagat gtcccctgtg ataatgtcag cagctgtccc tcctccgata |
| 6661 | cctgctgcca actcacgtct ggggagtggg gctgctgtcc aatcccagag gtatatggga |
| 6721 | ggggacagca tcttggcctg ggcaggtggg tggccaagct cctattgctt tctgccctcc |
| 6781 | gcatagccca taggtgatac ccagctctga cagattcgtc cccagctgga ggtgctgtaa |
| 6841 | gcaggagagg cgggctggag taggtagggg ctcggcactg cgccccacat agtggctacc |
| 6901 | tacaacgccc tttcctgccc accccccagg ctgtctgctg ctcggaccac cagcactgct |
| 6961 | gcccccaggg ctacacgtgt gtagctgagg ggcagtgtca gcgaggaagc gagatcgtgg |
| 7021 | ctggactgga gaagatgcct gcccgccggg cttccttatc ccaccccaga gacatcggct |
| 7081 | gtgaccagca caccagctgc ccggtggggc agacctgctg cccgagcctg ggtgggagct |
| 7141 | gggcctgctg ccagttgccc catgtgagtg cctccctgcc tgcccctgga taggggagct |
| 7201 | aagcccagtg aggggacagg aacataatgc cattctgtgc tcccttcccc gccaggctgt |
| 7261 | gtgctgcgag gatcgccagc actgctgccc ggctggctac acctgcaacg tgaaggctcg |
| 7321 | atcctgcgag aaggaagtgg tctctgccca gcctgccacc ttcctggccc gtagccctca |
| 7381 | cgtgggtgtg aaggacgtgg agtgtgggga aggacacttc tgccatgata accagacctg |
| 7441 | ctgccgagac aaccgacagg gctgggcctg ctgtccctac cgccaggtca gtgccaaccc |
| 7501 | ccatcctggg gctgggtatg gccagggacc aggtcccacc tcgtccaacc ctctcgcccc |
| 7561 | cctctgacca tccagggcgt ctgttgtgct gatcggcgcc actgctgtcc tgctggcttc |
| 7621 | cgctgcgcag ccaggggtac caagtgtttg cgcagggagg ccccgcgctg ggacgcccct |
| 7681 | ttgagggacc cagccttgag acagctgctg tgagggacag tactgaagac tctgcagccc |
| 7741 | tcgggacccc actcggaggg tgccctctgc tcaggcctcc ctagcacctc cccctaacca |
| 7801 | aattctccct ggaccccatt ctgagctccc catcaccatg ggaggtgggg cctcaatcta |
| 7861 | aggccttccc tgtcagaagg gggttgtggc aaaagccaca ttacaagctg ccatcccctc |
| 7921 | cccgtttcag tggaccctgt ggccaggtgc ttttccctat ccacaggggt gtttgtgtgt |
| 7981 | gtgcgcgtgt gcgtttcaat aaagtttgta cactttctta a |

### Example 2

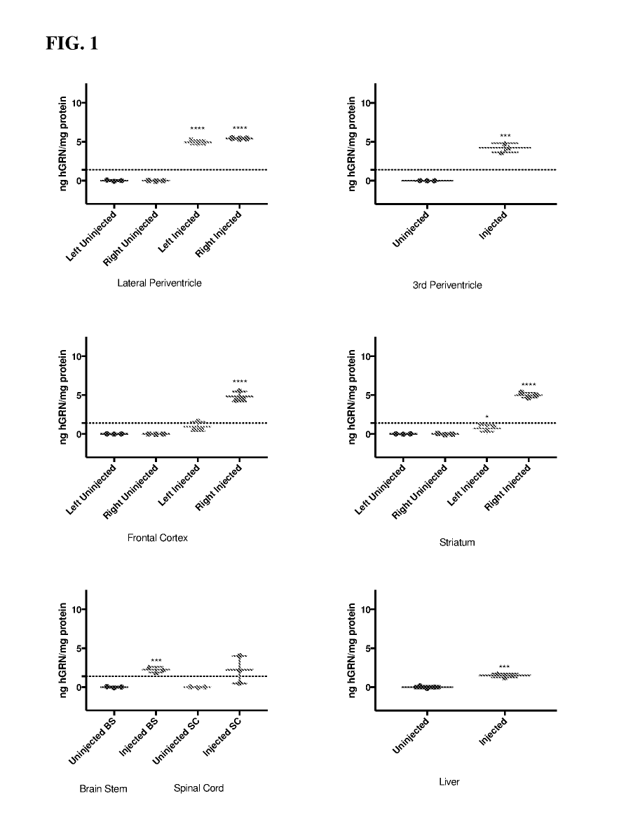

Human progranulin (hGRN) overexpression in the lateral periventricle, 3rd periventricle, frontal cortex, striatum, brain stem, spinal cord, and liver of GRN null mice 1 month post unilateral injection of 5E10 vg of AAV9.CMV.hGRN.bGHpA, compared to uninjected littermates, as measured by ELISA (FIG. 1). Dotted line indicates normal levels of hGRN in human frontal cortex as measured by ELISA. For lateral periventricle, frontal cortex, and striatum left and right indicate hemispheres of the brain, all mice were injected in the caudal right lateral ventricle.

### Example 3

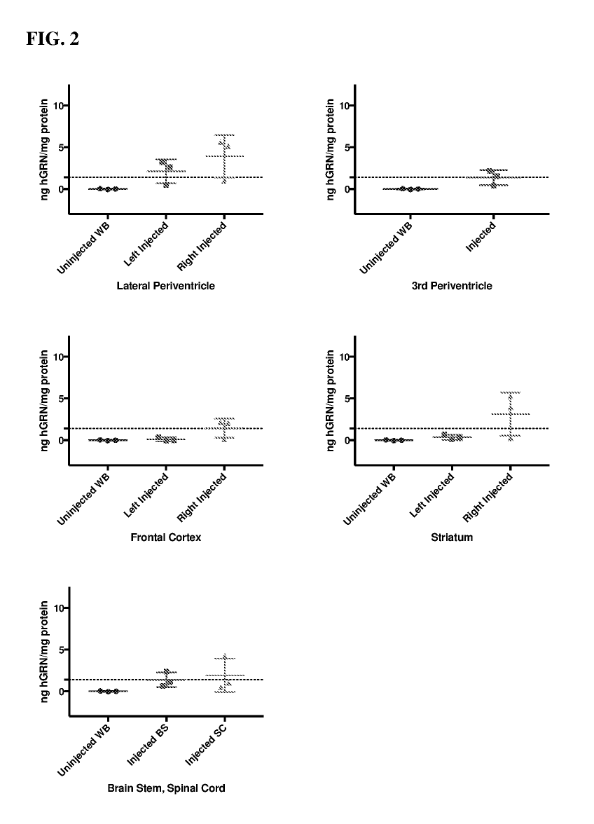

hGRN overexpression in the lateral periventricle, 3rd periventricle, frontal cortex, striatum, brain stem, and spinal cord of GRN null mice 3 month post unilateral injection of 5E10 vg of AAV9.CMV.hGRN.bGHpA compared to uninjected GRN-null whole brain (WB), as measured by ELISA (FIG. 2). Dotted line indicates normal levels of hGRN in human frontal cortex as measured by ELISA. For lateral periventricle, frontal cortex, and striatum left and right indicate hemispheres of the brain, all mice were injected in the caudal right lateral ventricle.

1. (canceled)

2. A method of treating a disease in a mammal caused by a deficiency or defect in progranulin expression or function, comprising administering to the mammal's brain ventricle an rAAV particle comprising a vector comprising a nucleic acid encoding progranulin, variant, derivative or functional fragment thereof in a manner effective to transduce cells that contact the cerebrospinal fluid (CSF) of the mammal, wherein the cell expresses the progranulin, variant, derivative or functional fragment thereof so as to treat the disease.

3. (canceled)

4. A method of treating a disease in a mammal caused by a deficiency or defect in progranulin expression or function comprising administering to the mammal's brain parenchyma, subarachnoid space and/or intrathecal space a vector comprising a nucleic acid encoding a progranulin, variant, derivative or functional fragment inserted between a pair of AAV inverted terminal repeats in a manner effective to transduce brain parenchyma cells or cells that contact the cerebrospinal fluid (CSF) of the mammal, wherein the cell expresses the progranulin, variant, derivative or functional fragment so as to treat the disease.

5. The method of claim 2, wherein the vector comprises a recombinant adeno-associated virus (rAAV) particle comprising an AAV capsid protein and the nucleic acid is inserted between a pair of AAV inverted terminal repeats.

6. The method of claim 5, wherein the AAV capsid protein is selected from the group consisting of AAV1, AAV2, AAV3, AAV4, AAV5, AAV6, AAV7, AAV8, AAV9, AAV10, AAV11, AAV12, AAV-rh74, AAV-rh10 and AAV-2i8 VP1, VP2 and/or VP3 capsid proteins, or a capsid sequence having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid sequences.

7. The method of claim 5, wherein the one or more of the pair of ITRs comprises or consists of an AAV1, AAV2, AAV3, AAV4, AAV5, AAV6, AAV7, AAV8, AAV9, AAV10, AAV11, AAV12, AAV-rh74, AAV-rh10 or AAV-2i8 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

8. The method of claim 2, wherein the vector further comprises an expression control element.

9. The method of claim 8, wherein the expression control element comprises a promoter or enhancer.

10. (canceled)

11. The method of claim 8, wherein the expression control element comprises a CMV enhancer, chicken beta actin promoter, CAG promoter and/or a sequence having 80% or more identity to CMV enhancer set forth in SEQ ID NO:4 and/or a sequence having 80% or more identity to CAG promoter set forth in SEQ ID NO:3.

12. The method of claim 5, wherein a plurality of rAAV particles are administered.

13. The method of claim 5, wherein the rAAV are administered at a dose of about 1×10⁶ to about 1×10¹⁸ vg/kg.

14.-18. (canceled)

19. The method of claim 2, wherein the delivering or administering comprises intraventricular injection.

20. (canceled)

21. The method of claim 2, wherein the brain ventricle comprises a lateral ventricle.

22. The method of claim 2, wherein the cells comprise ependymal, pial, endothelial, brain ventricle, meningeal, or glial cells and/or neurons.

23. (canceled)

24. The method of claim 2, wherein the cell secretes the progranulin, variant, derivative or functional fragment thereof into the CSF.

25.-27. (canceled)

28. The method of claim 2, wherein the vector is injected at 1-5 locations in the brain.

29.-41. (canceled)

42. The method of claim 2, wherein the method inhibits, decreases, or prevents neuron degeneration or death.

43.-44. (canceled)

45. The method of claim 2, wherein the method inhibits, decreases, or prevents cortical neuron degeneration or death.

46.-48. (canceled)

49. The method of claim 2, wherein the method improves, reduces or decreases a symptom or adverse effect of frontotemporal dementia (FTD) or Batten's disease.

50. The method of claim 2, wherein the method stabilizes, prevents worsening or reverses a symptom or adverse effect of frontotemporal dementia (FTD) or Batten's disease.

51.-54. (canceled)

55. The method of claim 2, wherein the mammal is human.

56. The method of claim 55, wherein the human is a child.

57.-60. (canceled)

61. The method of claim 2, wherein the disease comprises frontotemporal dementia (FTD) or Batten's disease.

62. The method of claim 2, further comprising administering one or more immunosuppressive agents.

63.-64. (canceled)
